(19) United States
(12) Patent Application Publication
(10) Pub. No.: US 2019/0015016 A1
(43) Pub. Date: Jan. 17, 2019

(54) WEARABLE ARTICLE

(71) Applicant: JKL IP COMPANY LLC, Sherman Oaks, CA (US)

(72) Inventors: Jodi E. Caden, Sherman Oaks, CA (US); Kari Kaden, Sherman Oaks, CA (US)

(21) Appl. No.: 15/958,953

(22) Filed: Apr. 20, 2018

Related U.S. Application Data
(63) Continuation-in-part of application No. 15/708,559, filed on Sep. 19, 2017.
(60) Provisional application No. 62/533,628, filed on Jul. 17, 2017.
(60) Provisional application No. 62/495,673, filed on Sep. 19, 2016.

(51) Int. Cl. A61B 5/11; A41D 1/00; A61B 5/00; G08B 21/04

(52) U.S. Cl. CPC A61B 5/1116; A41D 1/002; A61B 5/6804; A61B 5/7455; A41B 9/00; A61B 5/742; A61B 5/0002; A61B 5/4806; G08B 21/0446; A61B 5/7405

(57) ABSTRACT

A wearable article is provided. The wearable article may include a base portion, wherein the base portion may include a front portion, a back portion substantially opposite to the front portion, and an interior portion. The front portion may include at least one body section and the back portion may include at least one wedge section.

### CROSS-REFERENCE TO RELATED APPLICATIONS

This application is a continuation-in-part of U.S. patent application Ser. No. 15/708,559, filed Sep. 19, 2017, which claims priority to U.S. Provisional Application No. 62/533,628, filed Jul. 17, 2017, and U.S. Provisional Application No. 62/495,673, filed Sep. 19, 2016, all of which are hereby incorporated in their entireties by reference.

### TECHNICAL FIELD

Embodiments of the present invention generally relate to the field of wearable articles, such as garments or accessories, and more specifically to wearable articles that alert a wearer of an undesired body position.

### BACKGROUND

During the term of a pregnancy, especially in the second and third trimester, the blood flow and oxygenation to the fetus is crucial to its health. As the uterus grows larger it puts pressure on the large vessels, such as Vena Cava, in the pelvis. This decreases blood flow to the extremities. The Vena Cava is the main vein that carries blood back to the heart from the lower body. The aorta is the large artery that carries blood away from the heart to the lower body. After 20 weeks this compression can lead to smaller babies. It also decreases blood flow to the placenta, which affects the baby by depriving it of oxygen and essential nutrients. It also creates snoring and sleep apnea. Good sleep is very important to the expectant mother. The circulatory system of the mother and fetus allow the best oxygenation and blood flow when the mother lies on her left side. This flow may be compromised when the mother lies on her back, especially for a long period of time.

Studies on sleep position show that up to 25% of stillbirths could be prevented with lateral lie at night. The left side is most advantageous, but either side is preferable to supine positions. Encouraging pregnant women everywhere not to sleep on their back is a simple approach that may improve pregnancy outcomes.

Some expecting mothers place a pillow or a wedge foam against their side or under the uterus to display the uterus and/or to prevent rolling onto their back while sleeping. Or they rely on the person sleeping next to them to wake them up if they roll over. However, these systems are unreliable and neither prevent the expecting woman from rolling on her back nor wake her up if she does roll on her back.

Therefore, there is a need for a wearable article that ensures that an expecting mother does not lie on her back and also one that may alert an expecting mother upon such an event, thus leading to a safer environment in the utero and the ability to reduce the number of stillbirth rates up to 25% per current studies. The wearable article may also make it harder to turn to the supine position, thus preventing oxygen and aortocaval complications from being supine. The wearable article may also be used by other men or women wearers, who are not pregnant, to facilitate a better sleeping position to prevent snoring and sleep apnea.

### SUMMARY

Embodiments of the invention comprise a wearable article, such as a garment or accessory that ensures that a wearer's body is not in an undesirable orientation, such as supine. The wearable article may alert the wearer if such an event occurs. In some embodiments, the wearable article may include a base portion, wherein the base portion may include a front portion, a back portion substantially opposite to the front portion, and an interior portion. The front portion may include at least one body section and the back portion may include at least one wedge section.

This summary and the following detailed description are merely exemplary, illustrative, and explanatory, and are not intended to limit, but to provide further explanation of the invention as claimed. Other systems, methods, features, and advantages of the example embodiments will be or will become apparent to one skilled in the art upon examination of the following figures and detailed description.

### BRIEF DESCRIPTION OF THE FIGURES

The figures provided are diagrammatic and not drawn to scale. Variations from the embodiments pictured are contemplated. Accordingly, illustrations in the figures are not intended to limit the scope of the invention.

FIG. IA illustrates a front view of two inserts that can be inserted into a wearable article according to exemplary embodiments of the present invention.

FIG. IB illustrates a front view of two inserts that can be inserted into a wearable article according to exemplary embodiments of the present invention.

FIG. 1C illustrates a front view of two inserts that can be inserted into a wearable article according to exemplary embodiments of the present invention.

FIG. 2A illustrates a front view of a wearable article according to exemplary embodiments of the present invention.

FIG. 2B illustrates a back view of a wearable article according to exemplary embodiments of the present invention.

FIG. 2C illustrates a side view of a wearable article with an insert according to exemplary embodiments of the present invention.

FIGS. 2D, 2E, and 2F illustrate back views of a wearable article with inserts according to exemplary embodiments of the present invention.

FIG. 3A illustrates a front view of a wearable article according to exemplary embodiments of the present invention.

FIG. 3B illustrates a back view of a wearable article according to exemplary embodiments of the present invention.

FIG. 3C illustrates a back view of a wearable article with an insert according to exemplary embodiments of the present invention.

FIG. 3D illustrates a perspective view of an insert in a pocket according to exemplary embodiments of the present invention.

FIG. 3E illustrates a back view of a wearable article with an insert according to exemplary embodiments of the present invention.

FIG. 4A illustrates a front view of a wearable article according to exemplary embodiments of the present invention.

FIG. 4B illustrates a side view of a wearable article with an insert according to exemplary embodiments of the present invention.

FIG. 4C illustrates a back view of a wearable article with a pocket according to exemplary embodiments of the present invention.

FIG. 4D illustrates a back view of a wearable article with an insert according to exemplary embodiments of the present invention.

FIG. 4E illustrates a back view of a wearable article with a pocket according to exemplary embodiments of the present invention.

FIG. 4F illustrates a back view of a wearable article with an insert according to exemplary embodiments of the present invention.

FIG. 4G illustrates a back view of a wearable article with a pocket according to exemplary embodiments of the present invention.

FIG. 4H illustrates a back view of a wearable article with an insert according to exemplary embodiments of the present invention.

FIGS. SA and SB illustrate a wearable article with an insert according to exemplary embodiments of the present invention.

FIG. SC illustrates a back view of a wearable article with a pocket according to exemplary embodiments of the present invention.

FIG. SD illustrates a front view of a wearable article according to exemplary embodiments of the present invention.

FIG. SE illustrates a back view of a wearable article according to exemplary embodiments of the present invention.

FIG. SF illustrates a front view of a wearable article according to exemplary embodiments of the present invention.

FIG. 6A illustrates a front view of a wearable article according to exemplary embodiments of the present invention.

FIG. 6B illustrates a side view of a wearable article with a pocket according to exemplary embodiments of the present invention.

FIG. 6C illustrates a back view of a wearable article with an insert according to exemplary embodiments of the present invention.

FIG. 6D illustrates a perspective view of an insert in a pocket according to exemplary embodiments of the present invention.

FIG. 7 illustrates a schematic view of a wearable article communicatively linked with mobile computing devices according to exemplary embodiments of the present invention.

FIG. 8A illustrates a top view of a wearable article according to exemplary embodiments of the present invention.

FIG. 8B illustrates a side view of a wearable article according to exemplary embodiments of the present invention.

FIG. 8C illustrates a top view of a wearable article according to exemplary embodiments of the present invention.

FIG. 8D illustrates a top view of a portion of a wearable article according to exemplary embodiments of the present invention.

FIG. 8E illustrates a top view of a wearable article according to exemplary embodiments of the present invention.

FIG. 8F illustrates a top view of a wearable article according to exemplary embodiments of the present invention.

### DETAILED DESCRIPTION

The following disclosure describes various embodiments of the present invention and method of use in at least one of its preferred, best mode embodiment, which is further defined in detail in the following description. Those having ordinary skill in the art may be able to make alterations and modifications to what is described herein without departing from its spirit and scope. While this invention is susceptible to different embodiments in different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

In the following description and in the figures, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

The disclosure herein describes wearable articles including but not limited to garments and/or accessories that include but are not limited to undergarments, garments worn on the top half of the body, garments worn on the lower half of the body, garments that cover both halves of the body, accessories, such as belts, watches, wraps, pillows, devices, equipment, etc. Wearable article as used herein means any garment or accessory that can be put on, attached to, stuck on, applied on, etc. a wearer. It may also mean any garment or accessory into which a wearer can enter, lie in or on, surround himself or herself in or with, etc.

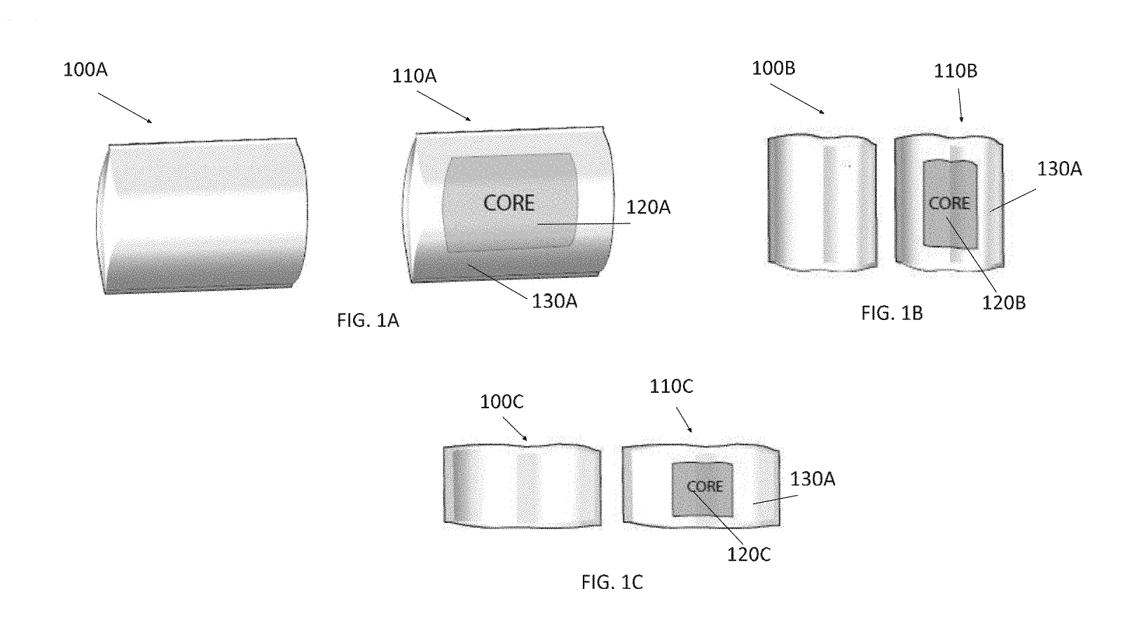

FIGS. 1A-IC illustrate exemplary embodiments of inserts IOOA, 11OA, IOOB, 11OB, 1OOC, and 11OC that can be inserted into a wearable article, such as those illustrated in the following figures. As illustrated in FIG. 1A, the insert IOOA and 11OA may have a substantially lumbar shape that is oriented horizontally. In some embodiments, the insert IOOA may be made of a uniform material throughout. In other embodiments, the insert 11OA may be made of two or more materials. As illustrated in FIG. 1A, the insert 11OA may have different composition of materials in the outer periphery 130A and the core 120A. The core 120A may be made of a material that is firmer than the outer periphery's 130A material or vice versa. The inserts may have any other shape and/or orientation as well. For example, in some embodiments inserts IOOB and 11OB illustrated in FIG. 1B have a substantially hourglass shape oriented vertically. In other embodiments, the inserts 1OOC and 11OC illustrated in FIG. 1C have a substantially horizontal orientation. Various materials that may be used to make the insert, its outer periphery, and/or its core may include the following non-limiting examples: any organic, natural, man-made, or synthetic materials, such as charcoal foam, polyethylene foam, memory foam, anti-static foam, rebond foam, gel, silicone, rubber, sponge, feathers, contour foam, polyester fiberfill, microbeads, cotton, wool, latex, pvc, microbeads, memory foam, bird feathers, etc. In some embodiments, the insert may be any balls, such as tennis balls and footballs, fabrics rolled up, etc. In some embodiments, the inserts may comprise a shape that automatically shifts the body of the wearer from an undesirable position to a desirable position. In other embodiments, the inserts may comprise features as described below in FIG. 7. Each embodiment of the insert 100A, 11OA, 1OOB, 11OB, 1OOC, and 11OC described above and in the following figures may be capable of being placed within the article in any way. For example, in some embodiments, one or more inserts 1OOA, 11OA, 1OOB, 11OB, 1OOC, and 11OC may be placed in one or more pockets located anywhere on the article. In some embodiments, one or more inserts 1OOA, 110A, 1OOB, 11OB, 1OOC, and 11OC may be placed between the article and the wearer. In some embodiments, one or more embodiments may be placed between the skin of the wearer and article. In some embodiments, 1OOA, 11OA, 1OOB, 11OB, 1OOC, and 11OC one or more inserts may be embedded within the article. The inserts 1OOA, 11OA, 1OOB, 11OB, 1OOC, and 11OC and other inserts describe below may of any shape and/or size as well. For example, in some embodiments, the inserts 1OOA, 11OA, 1OOB, 11OB, 1OOC, and 11OC and other inserts describe below may be of a substantially lumbar, hourglass, cuboidal, spherical, triangular, etc., shape.

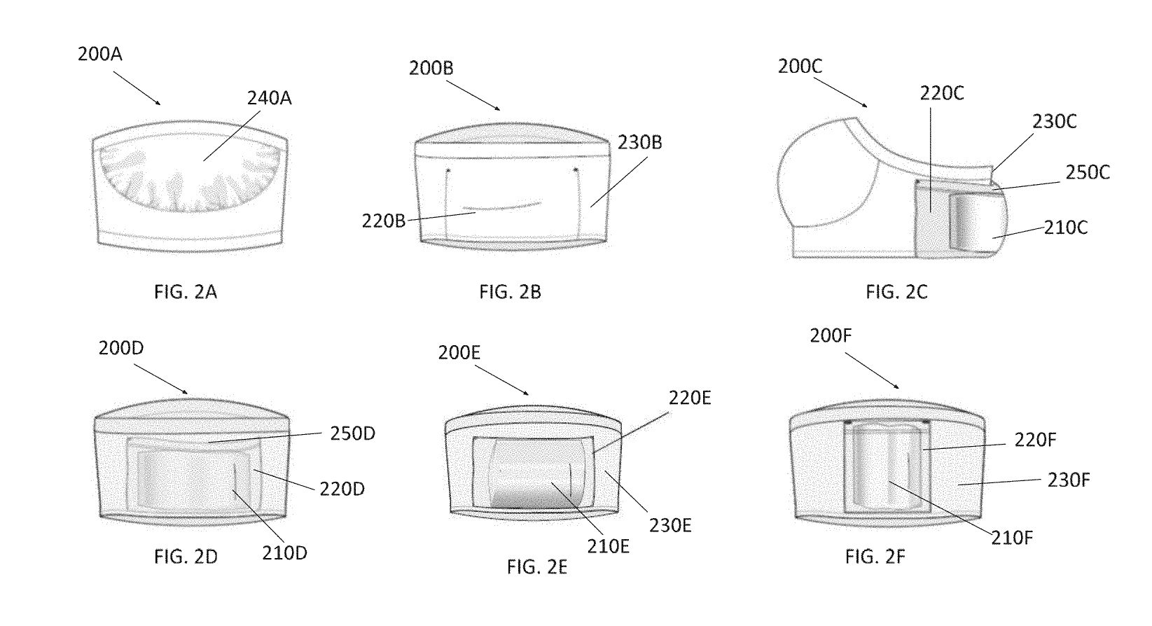

FIGS. 2A-2F illustrate wearable articles 200A, 200B, 200C, 200D, 200E, 200F, such as the Belly Boost™ wrap from Belly Bandit®. As illustrated in FIG. 2A, the wearable article 200A may have a stretchable pocket 240A to accommodate various body parts of the wearer, such as their stomachs, breasts, etc. The article 200A may have a front portion 210A and a side portion 220A. In some embodiments, as illustrated in FIG. 2B, the wearable article 200B may include a pocket 220B at the back portion 230B of the wearable article 200B. The pocket 220B may be completely transparent, partially transparent, or completely opaque. As illustrated in FIG. 2C, the pocket 220C may completely cover the back portion 230C of the wearable article 200C. In some embodiments, the pocket 220C may only cover a part of the back portion 230C. In other embodiments, there may be one or more pockets placed at different locations of the wearable article 200C. In some embodiments, as illustrated in FIG. 2D, the pocket 220D may have at least one opening 250D so that an insert 21OD may be housed in and removed from the pocket 220D at will. In some embodiments, the opening 250D may be oriented along the bottom seam of the sleeve 220D. In other embodiments, the opening 250D may be on any side. There may also be more than one openings 250D in the pocket 220D. As illustrated in FIG. 2E, the pocket 220E may not have any opening. An insert 210E may already be placed in the wearable article 200E and sealed within the pocket 220E on the back 230E of the wearable article 200E. As illustrated in FIG. 2F, an insert 210F may be aligned substantially vertically within a pocket 220F. In some embodiments, each pocket described above, may be capable of housing more than one insert of any shape, characteristics, and orientation.

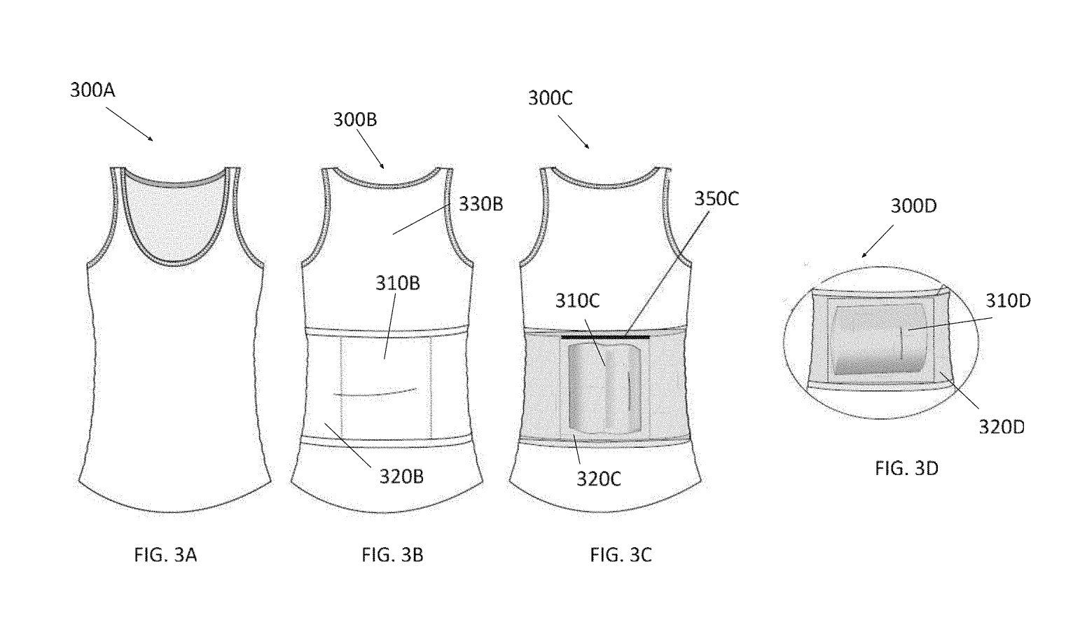

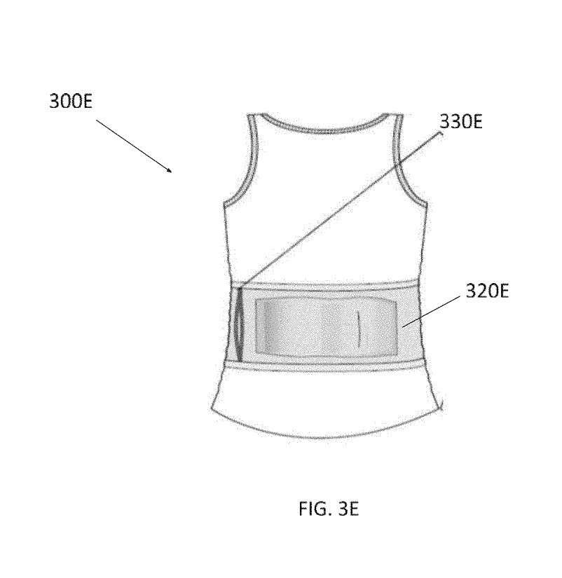

FIGS. 3A-3C and 3E illustrate wearable articles 300A, 300B, 300C, and 300E, such as a tank top. In some embodiments, the article 300A may have a front portion 310A and a side portion 320A. In some embodiments, as illustrated in FIG. 3B, the wearable article 300B may include a pocket 320B at the back portion 330B of the wearable article 300B. The pocket 320B may be completely transparent, partially transparent, or completely opaque. The pocket 320B may only cover a portion of the back portion 330B or may completely cover the back portion 330B of the wearable article 300B. In some embodiments, the pocket 320B may only cover a part of the back portion 330B. In some embodiments, the pocket 320B may be located on the side portion. In other embodiments, there may be one or more pockets placed at different locations of the wearable article 300B. In some embodiments, as illustrated in FIG. 3C, the pocket 320C may have at least one opening 350C so that an insert 310C may be housed in the pocket 320C and removed at will. The insert 310C illustrated in FIG. 3C is oriented in a vertical direction. However, the insert may be oriented in any direction. For example, as illustrated in FIG. 3D, the insert 310D may be housed in an essentially horizontal manner within the pocket 320D. Also, the pocket 320D may not have any openings. The insert 310D may be inserted into the pocket 320D at any stage and the sealed shut within. In other embodiments, the opening 350C may be on any side. For example, as illustrated in FIG. 3E, the opening 330E may be located on the side of the pocket 320E. In some embodiments, each pocket described above, may be capable of housing more than one insert of any shape and orientation.

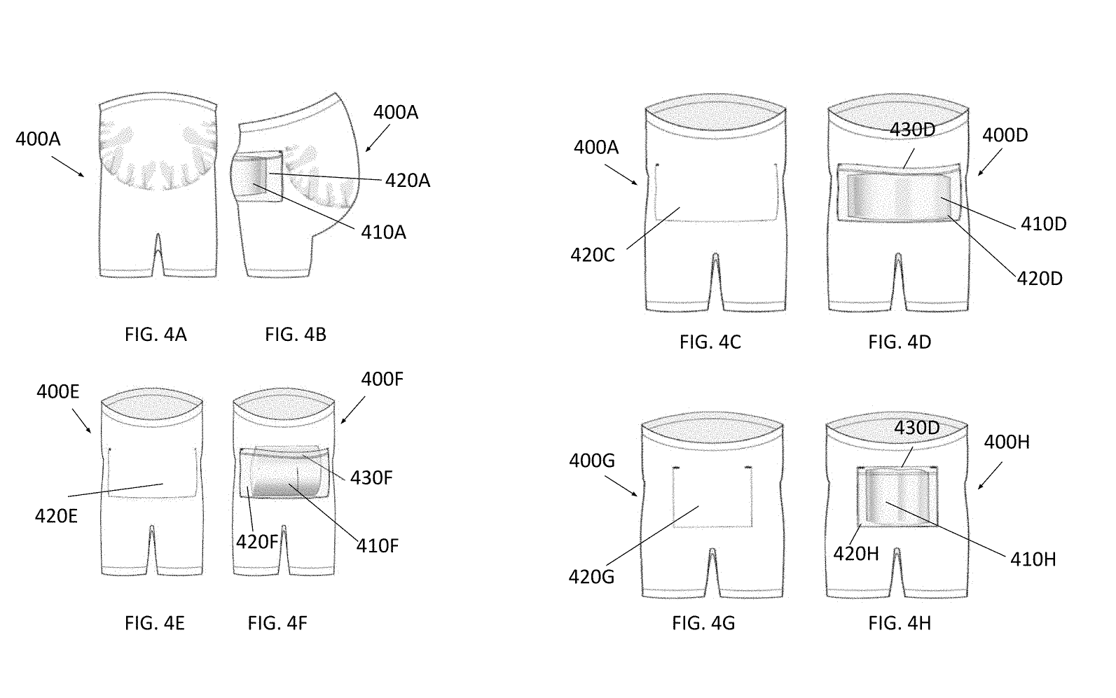

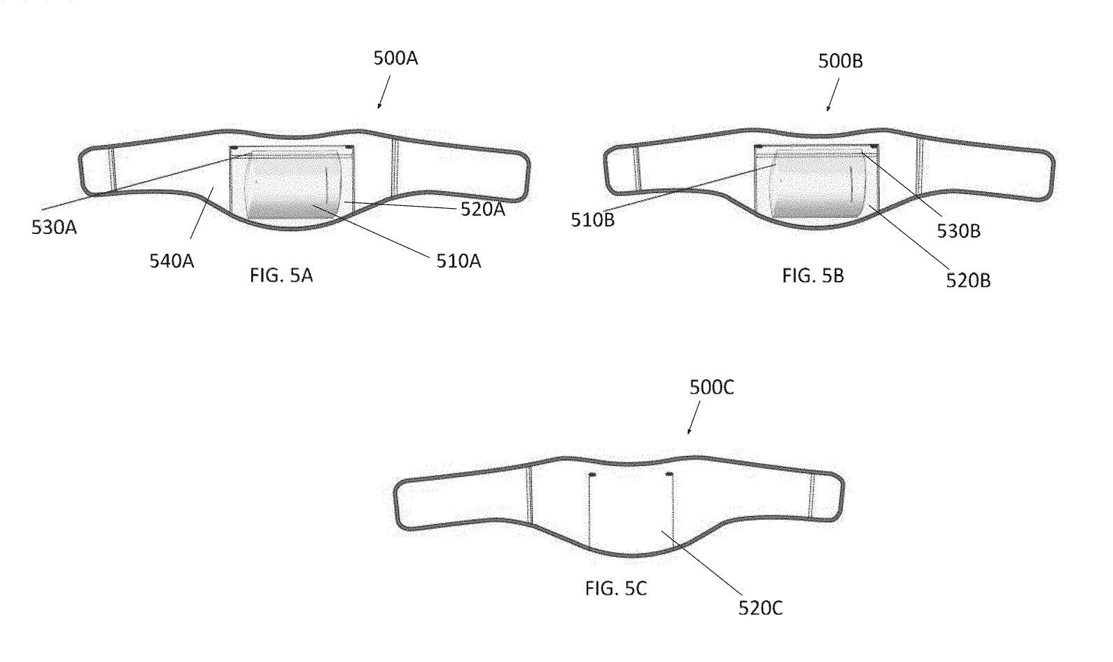

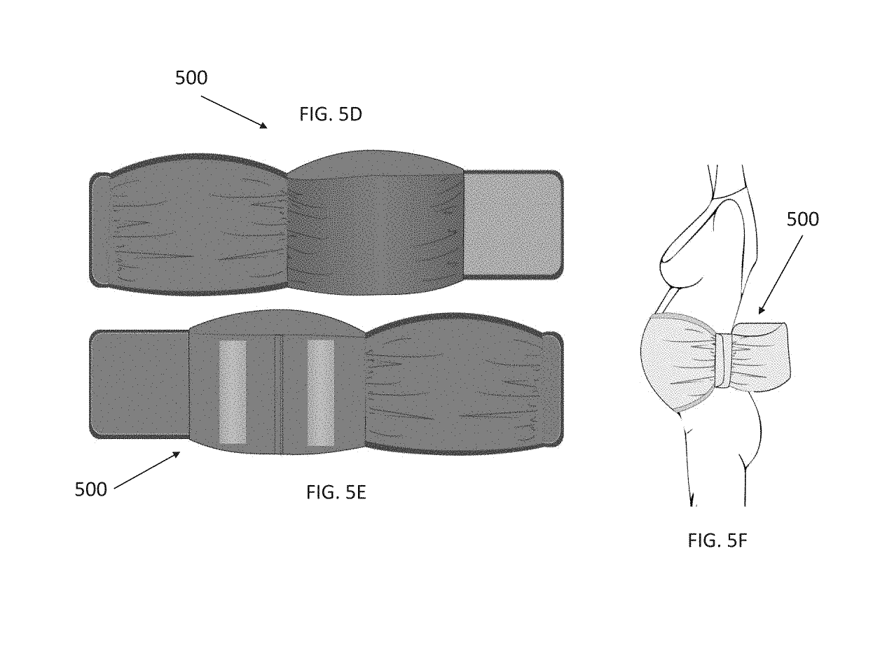

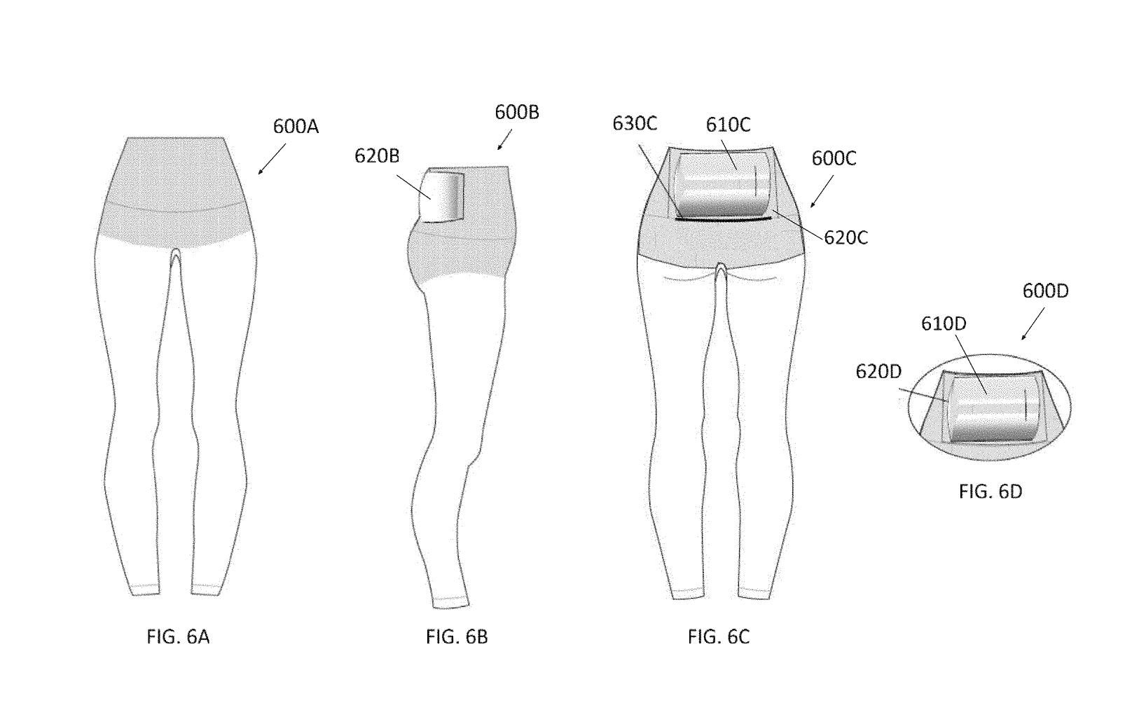

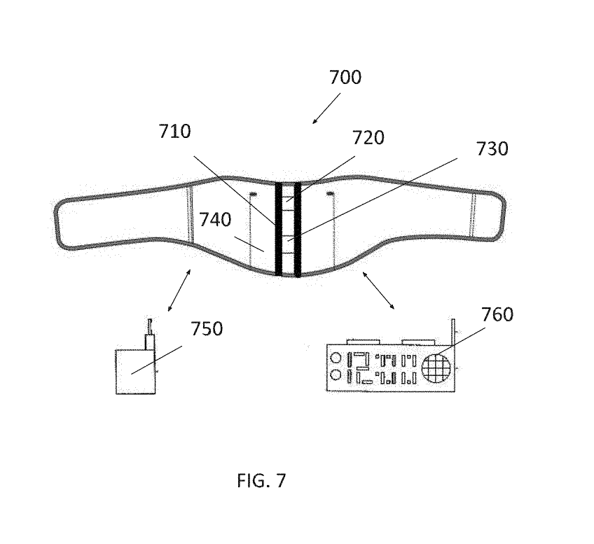

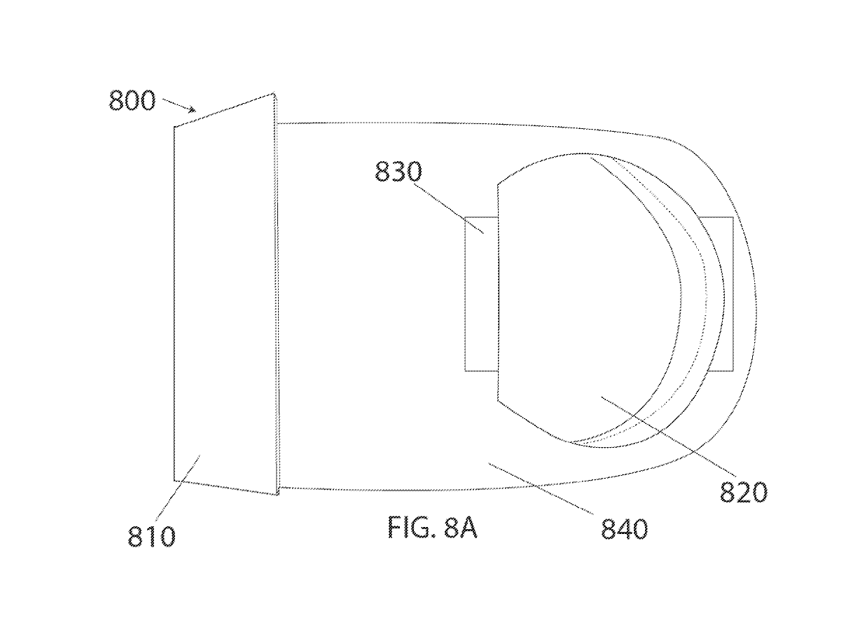

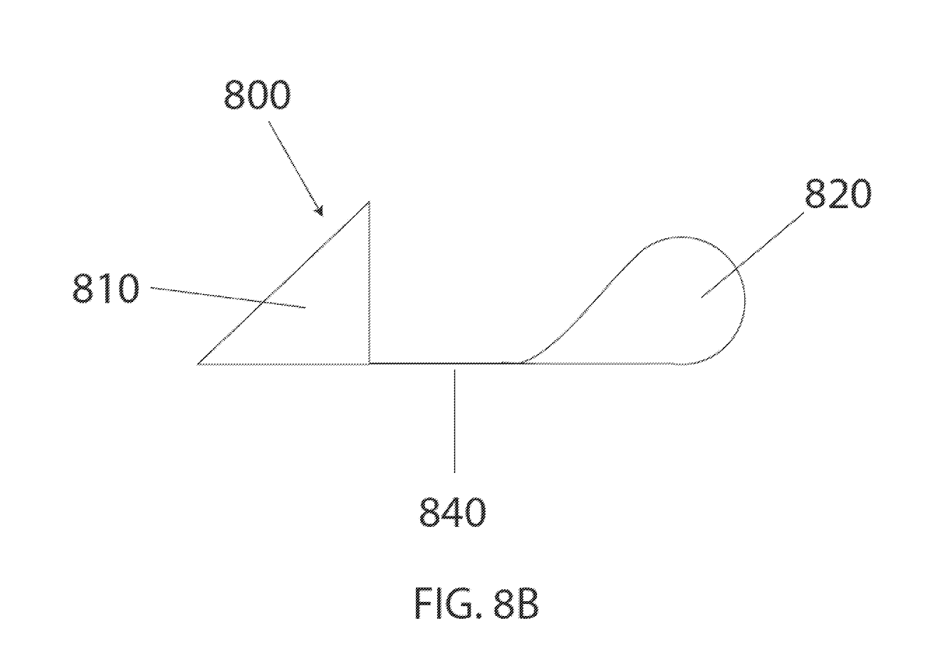

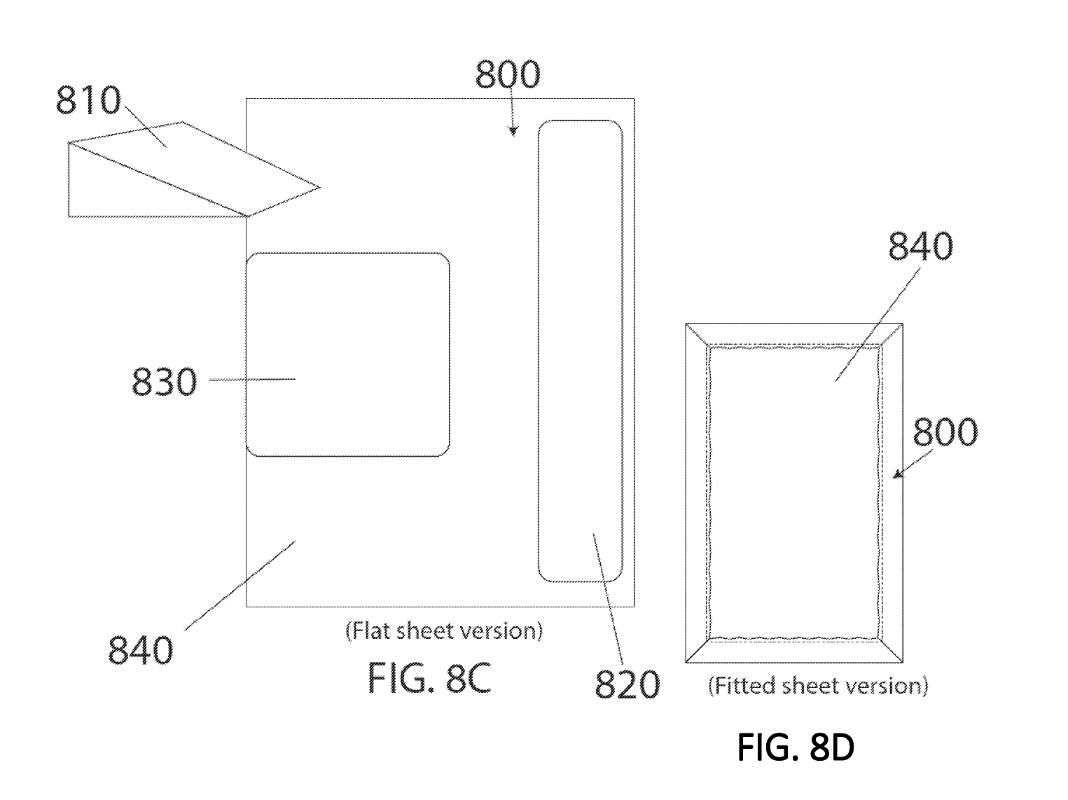

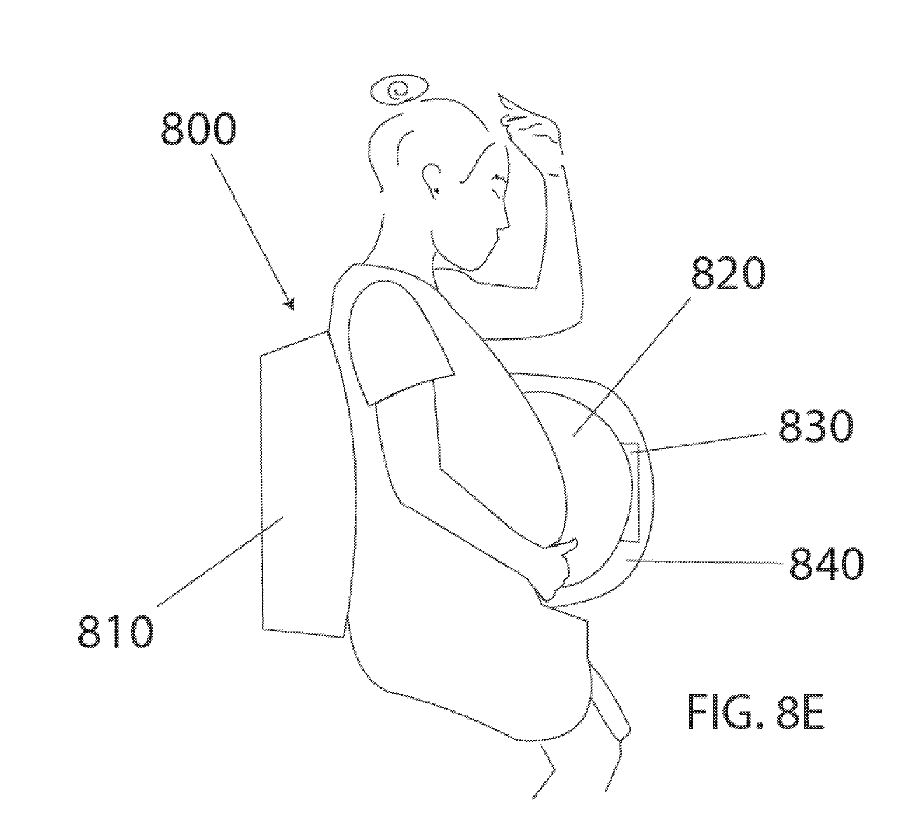

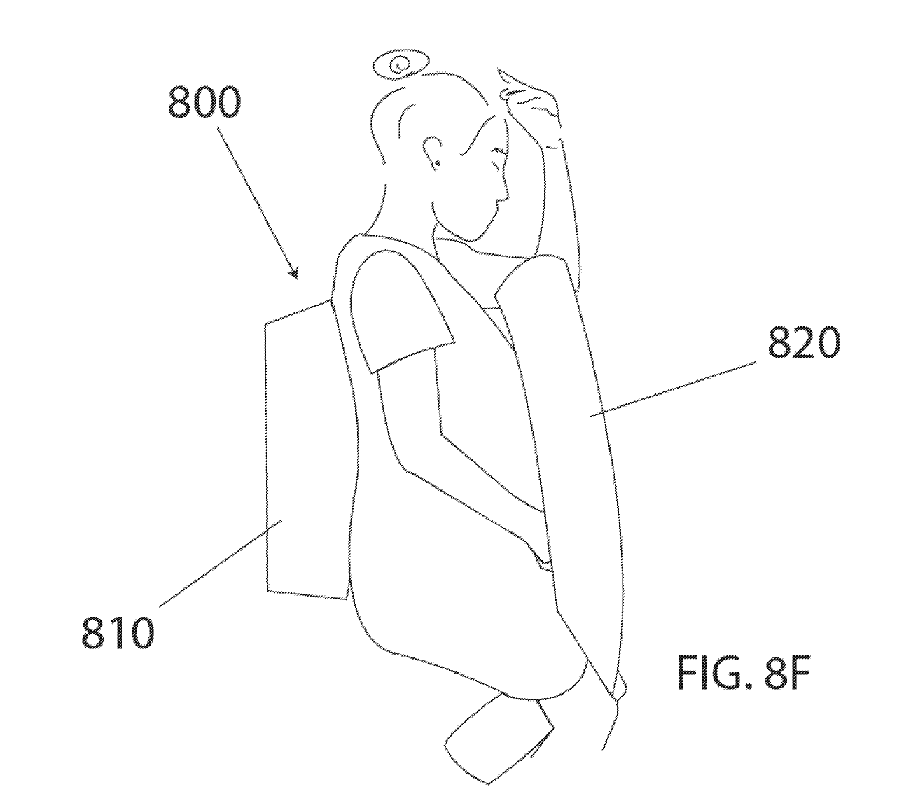

FIGS. 4A-4H illustrate a wearable article 400A, 400B. 400C, 400D. 400E. 400F, 400G, and 400H such as the Thighs Disguise and/or Bump Support1M by Belly Bandit®. As illustrated in FIGS. 4F and 4H, the pocket 420F may completely cover the back portion of the wearable article 400F or the pocket 420H may only cover a portion of the back portion of the wearable article 400H. FIGS. 6A-6C illustrate a wearable article 600A. 600B, and 600C, such as the Bump Support1M and/or B.D.A™ leggings by Belly Bandit®. The wearable articles illustrated in FIGS. 4A-4H and 6A-6C have similar properties to the wearable articles described in FIGS. 2A-2F. FIG. 6D illustrates a pocket 620D that is sewn shut and has no opening to remove the insert 610D. In some embodiments, it has similar properties as described in FIG. 3D.

FIGS. 5A-5C illustrate a wearable article 500A. 500B. 500C, and 500D, such as Belly Wraps by Belly Bandit®. In some embodiments, as illustrated in FIG. SA, the wearable article 500A may include a pocket 520A at its back portion 540A. The article 500A may have a front portion 510OA and a side portion 530A. The pocket 520A may be completely transparent, partially transparent, or completely opaque. The pocket 520A may completely cover the back portion 540A of the wearable article 500A. In some embodiments, the pocket 520A may only cover a part of the back portion 540A. In other embodiments, there may be one or more pockets placed at different locations of the wearable article 500A. In some embodiments, the pocket 520A may have at least one opening 530A located on the top so that an insert 510A may be housed in the pocket 520A and removed at will. In other embodiments, the opening 530A may be on any side of the pocket 520A. The insert 51OA may be housed in any direction and may be of any shape. In some embodiments, more than one insert may be housed within each pocket. As illustrated in FIG. 5B, the pocket 520B may not have any openings. The insert 51OB may be inserted into the pocket 520B at any stage and the sealed shut by any means, such as sewing. As illustrated in FIG. 5C, the wearable article 500A may have a front portion 51OC, a back portion 520C, and a side portion 530C. In some embodiments, the wearable article 500 may have dimensions and aesthetics as illustrated in FIGS. 5D-5F. The wearable article 500 may be able to opens/closes at the side with hook and loop or any other attachment mechanism. It may also be cut and sewn. It may have seams. It may be made with breathable materials.

FIG. 7 illustrates a wearable article 700, such as Belly Wraps by Belly Bandit®. The article 700 may have a front portion 780 and a side portion 770. The wearable article 700 may comprise a pocket 740. The pocket may have at least one opening or it may be sealed shut. The pocket 740 may house an insert 710. The insert 710 may comprise at least one force-measuring sensor 720 and a transmitter 730. The force-measuring sensor 720 may be of any kind, such piezo resistive, thin-film strain gauge, conductive, piezoelectric, printed circuitry, etc. The measuring sensor 720 may generate signals based on the amount of force exerted on it when the orientation of the body of the wearer shifts. In some embodiments, the force-measuring sensors 720 may be isolated pieces and may be placed in predetermined positions throughout the wearable article 700. Additionally, in some embodiments, the wearable article 700 may include features that allow for it to be remotely accessed and monitored. For example, a mobile computing device 750, such as a tablet computer, laptop, smart phone, or wearable computing device, may also be communicatively coupled to the wearable article 700 through a wired or wireless communication link, such as Bluetooth. Internet, a wide area network, a local area network, a metropolitan area network, a virtual private network, a cellular network, or any other type of wired or wireless network. The mobile computing device 750 may be configured to send and receive signal to and from the wearable article 700 about when a wearer's body is in an undesirable orientation. This may facilitate remote monitoring of the orientation of the wearer's body. For example, when the wearer exerts enough force on the sensor 710 by laying on her back, the sensor 710 may generate a signal that indicates that a threshold force has been met. This signal may be transmitted to the mobile computing device 750 using the transmitter 730. The mobile computing device 750 may be capable of displaying the signal or generating some signal upon receiving the transmitted signal. In some embodiments, based upon the received signal, the wearer of the mobile computing device may either be able to shift the position of the wearer manually. In other embodiments, the mobile computing device may be capable of generating a reaction within the wearable article 700, such as generating a heat signal, vibration, sounds, etc., that will alert the wearer that they are oriented in an undesirable position. The wearable article 700 may also include features that allow temperature manipulation. The wearable article 700 may also include features that may replicate a massaging effect for the wearer. In other embodiments, the undesired position may be pre-set by the wearer. For example, the wearer may have an injury on the ribs and sleeping on the side may not be recommended. Therefore, the wearable article 700 may be programmed to alert the wearer that he or she is sleeping on the side. In some embodiments, the wearable article 700 may be communicatively linked to other devices 760, such as the TV, radio, clock, etc., such that the wearer may operate the devices remotely while wearing the wearable article 700. The wearable article 700 may also contain features that allow pregnant mothers to determine the health and progress of the baby and/or the mother, including but not limited to heart rate, blood pressure, etc., while they are wearing the garment. The wearable article 700 may also be capable of syncing to a hospital service such that the hospitals may perform real-time monitoring of the condition of the wearer or in the case of the pregnant mother also her child. This may allow the hospital to be on notice in case of an emergency while the wearer is wearing the garment. The wearable article 70 may also contain heat emitters, sounds emitters, light emitters, and/or vibrators to alert the wearer of the position of their body based on the signal generated by the sensors 720.

The transmitter 730 may transmit the sensor data in any manner, such as wirelessly, as described above. The insert 710 may also comprise a memory, a processing unit, a local computing device a charging port, and/or a display screen. The memory may store all the data generated by the sensors 720 or just some data that meets a predetermined threshold force. In some embodiments, the wearable article 700 may also contain a location based positioning system. In some embodiments, the wearable article 710 may a power source, such as a battery. Additionally, a switch may be coupled with the power source for turning the sensor 710 on and off In some embodiments, the pocket 740 may only house one insert 710. In other embodiments, the pocket 740 may house more than one insert 710. In other embodiments, other inserts, such as the ones described in the previous FIGs, may be combined with the insert 710. In other embodiments, insert 710 may be located within the inserts described in the previous FIGS. The pocket 740 may have at least one opening located on any side or it may be sealed shut. In some embodiments, the wearable article may have other smart features, such as those described in http://www.owletcare.com/ that allow tracking the wearer's health while they are wearing the wearable article. Features described by Owletcare on their website are incorporate by reference herein in their entirety. It may also have the alarm features described in in U.S. Patent Publ. No. 2010/0201526, which is incorporated by reference herein in its entirety.

FIGS. 8A-8F illustrates a wearable article 800. The article 800 may include a wedge section 810, such as a pillow, foam piece, bean bag, silicone, plastic, rubber, etc. The wedge section 810 may be made of, filled with, or covered in any material as desired, such as cotton, polyester, foam, feathers, wool, silk, etc. The wedge section 810 may be attached to a base portion 840 on its front or back portion using any attachment mechanisms 830, such as Velcro®, adhesive, tape, sowing, snaps, buttons, magnets, screws, microfiber silent Velcro® loop, etc. The base portion 840 may be of any size and shape, such as hourglass, semi-circular, rectangular, square, round, crescent, moon, “S.” triangle, oval, etc. It may also fit within the measurement of—or fit on a twin, full, queen, and/or king-sized bed. The article 800 may also include a body section 820 on its front or back portion. The body section 820 may be of a different shape and size than the wedge section 810. The body section 820 may also be attached to the base portion 840 using any of the aforementioned attachment mechanisms. The wedge section 810 or the body section may be detachable from the base portion 840. The base portion 840 (or other sections 810 and 820) may be made of any material, such as wool, silk, silicon, rubber, memory foam, etc. The wedge portion 810 may be configured to support the back of a wearer as illustrated in FIGS. 8E and 8F. The body portion 820 may be configured to support the front portion, such as the belly, of the wearer as illustrated in FIGS. 8E and 8F. The wearer may enter into the interior portion of the wearable article 800 such that he or she can comfortably lie in there. The base portion 840 may have a gripper section, such as Velcro® sections, gripper dots, etc. The sections 810 and 820 are not limited in number. For example, the body section 820 may have multiple sections located next to each other. The body section 820 may be of any shape, such as hourglass, semi-circular, rectangular, square, round, crescent, moon, “S.” triangle, oval, etc., and size as well. In some embodiments, the section 810 and 820 may be a unitary piece or two pieces that can be attached to each other to form a unitary piece. Such a unitary piece may then also be able to be attached to the base portion 840. The sections 810 and 820 may be of the larger, equal to, or shorter, than each other and/or the base portion 840.

The wearable articles are not limited to the embodiments described above. For example, the articles may be wearable, such as shorts, t-shirts, shirts, bras, skirts, pants, girdles, belly bands, belts, tank tops, tube tops, nightgowns, etc. The articles may be made of any one or combination of manmade, natural, or synthetic materials, such as bamboo, spandex, cotton, neoprene, polyester/spandex, nylon/spandex, elastane, Tencel® viscose, rayon, silk, satin, nylon/cotton, microfiber, Lycra®, elastic, rubber, acetate, cotton, denim, etc. The pockets described above may be located on the interior or the exterior side of the articles. The pockets may also pass through the interior and the exterior side of the wearable articles. The pockets may have seams or be seamless. They may be made using any techniques, such as cutting and sewing, knitting, crocheting, etc. The pockets may have pockets within them to house additional inserts or any other items. The pockets may have openings on any one or more sides or they may be sealed shut by sewing, buttons, magnets, clasps, snaps, tacking, hooks, Velcro®, zippers, etc. The pockets may be detachable from the articles. The inserts may be made using any known techniques. One or more inserts of any type may be inserted into the same articles. Further, the articles may also comprise articles that can be used to achieve similar purposes in any other way. The articles described in various embodiments may have features described in any figures. Thus, features described in any embodiment may be used interchangeably in other embodiments.

1. A wearable article comprising:
a base portion;
wherein the base portion comprises a front portion and a back portion substantially opposite to the front portion;
wherein the front portion comprises at least one body section and the back portion comprises at least one wedge section; and
an interior portion.

2. The wearable article of claim 1, wherein at least one body section is permanently fixed to the base portion.

3. The wearable article of claim 1, wherein at least one wedge section is permanently fixed to the base portion.

4. The wearable article of claim 1, wherein body section is removable from the base portion.

5. The wearable article of claim 1, wherein at least one wedge section-is removable from the base portion.

6. The wearable article of claim 1, wherein at least one wedge section has a tapering width.

7. The wearable article of claim 1, wherein at least one wedge section has a length extending the whole length of the base portion.

8. The wearable article of claim 1, wherein at least one wedge section has a length extending only a portion of a back portion of the wearer.

9. The wearable article of claim 1, wherein at least one body section has a length extending the whole length of the base portion.

10. The wearable article of claim 1, wherein at least one wedge section has a length extending only to a front portion of a wearer operable to provide support to a belly portion of a wearer.

11. The wearable article of claim 1, wherein the at least one wedge section is attached to the base portion using an attachment mechanism.

12. The wearable article of claim 1, wherein the at least one wedge section has at least one of a lumbar, hourglass, substantially triangular, semi-circular, rectangular, round, square, crescent shape, moon shape, S-shape, and oval shape.

13. The wearable article of claim 1, wherein at least one of a base portion, wedge section, and body section comprises a force-measuring sensor.

14. The wearable article of claim 1, wherein at least one of a base portion, wedge section, and body section comprises a motion-measuring sensor.

15. The wearable article of claim 1, wherein at least one of a base portion, wedge section, and body section comprises at least one of a heat emitter, sound emitter, and vibration emitter.

16. The wearable article of claim 1, wherein at least one of a base portion, wedge section, and body section comprises a light emitter.

17. The wearable article of claim 1, wherein at least one of a base portion, wedge section, and body section comprises a transmitter.

18. The wearable article of claim 1, wherein at least one of a base portion, wedge section, and body section comprises a power source.

19. The wearable article of claim 1, wherein at least one of a base portion, wedge section, and body section comprises a sensor to monitor the health of the wearer.

20. The wearable article of claim 1, wherein the body section has at least one of a substantially lumbar, semi-circular, rectangular, round, hourglass, square, crescent, moon, S, triangular, and oval shape.
